(19) United States
(12) Patent Application Publication
(10) Pub. No.: US 2024/0341965 A1
(43) Pub. Date: Oct. 17, 2024

(54) DISK-SHAPED AUGMENTATION FOR A BONE, PARTICULARLY A LONG BONE

(71) Applicant: Waldemar Link GmbH & Co. KG, Hamburg (DE)

(72) Inventor: Helmut D. LINK, Hamburg (DE)

(73) Assignee: Waldemar Link GmbH & Co. KG, Hamburg (DE)

(21) Appl. No.: 18/683,271

(22) PCT Filed: Aug. 16, 2022
(86) PCT No.: PCT/EP2022/072851

§ 371 Date: Feb. 13, 2024

(30) Foreign Application Priority Data
Aug. 16, 2021 (EP) 21000233.3
Aug. 8, 2022 (WO) PCT/EP2022/072233

(51) Int. Cl. A61F 2/38; A61F 2/30

(52) U.S. Cl. CPC A61F 2/389; A61F 2002/30113; A61F 2002/30433; A61F 2002/30736; A61F 2002/3092

(57) ABSTRACT

Disk-shaped augmentation for filling bone defects, in particular at the end of long bones, such as the tibia, including a first side, a second side, an outer sheath on lateral sides and an inner wall for a through-opening, running from the first to the second side, for a anchoring keel of an endoprosthesis arranged on the second side. The augment is generally C-shaped with two legs flanking the through-opening. According to the invention, a connecting piece between the legs is designed in an articulated manner and cooperates resiliently with the legs such that a frame is formed, and an outwardly directed restoring force is generated when the legs are compressed.

### CROSS-REFERENCE TO RELATED APPLICATIONS

This application is a National Stage under 35 U.S.C. 371 of International Patent Application No. PCT/EP2022/072851, filed Aug. 16, 2022, which claims priority to: (i) European Patent Application No. 21000233.3, filed Aug. 16, 2021; and (ii) International Patent Application No. PCT/EP2022/072233, filed Aug. 8, 2022; the disclosures of all of which are incorporated herein by reference in their entirety.

### FIELD

The invention relates to a disk-shaped augment for filling bone defects, particularly at the end of long bones such as the tibia. The augment is provided with a first side, a second side, an outer sheath on lateral sides and an inner wall for a through-opening running from the first to the second side for an anchoring keel of an endoprosthesis arranged on the second side.

### BACKGROUND

When implanting endoprostheses, especially joint endoprostheses, a problem sometimes arises in that the bone receiving the endoprosthesis is damaged, particularly in the area of the end of the bone (bone head). The reason for this is, in particular, defects in the (spongy and/or cortical) bone substance or surface, e.g., due to illness or injury, but increasingly also due to the explantation of an older prosthesis. In order to still create a sufficient base in the bone to anchor the endoprosthesis, augments are typically used to fill in the missing bone substance. It has proven useful to arrange them sunk into the end of the bone so that they are surrounded like a quiver by the cortical edge of the end of the bone.

Disk-like augments for use with the tibial component of a knee joint endoprosthesis are known, for example, from EP 1 360 950 B1. Two half-sided augments are provided below the tibial plate, which are arranged to the left and right of an anchoring keel. The augments abut flush against the bottom of the tibial plate and are, in the assembled state, secured there with a fastening screw. It has been shown that in this way the augments are held in a defined manner in relation to the endoprosthesis, but not in relation to the bone head that they actually are supposed to support or whose bone defect they are supposed to fill. A stronger reference to the bone defect or to the space to be filled in the bone defect would be desirable. A further difficulty can arise after long-term use because the augment grows into the bone over time and the endoprosthesis with the augment is then difficult or almost impossible to remove, for example as part of revision surgery. There is a risk of further damage to the already sensitive bone head.

The invention is based on the object of creating an improved augment with which the aforementioned disadvantages can be avoided or reduced.

The solution according to the invention is found in the features of the independent claim. Advantageous refinements are the subject of the dependent claims.

### SUMMARY

In a disk-shaped augment for filling bone defects, in particular at the end of long bones such as the tibia, with a first side, a second side, an outer sheath on lateral sides and an inner wall for a through-opening running from the first side to the second side for an anchoring keel of an endoprosthesis arranged on the second side, wherein the augment has a generally C-shaped shape with two legs flanking the through-opening, according to the invention it is provided that a connecting piece between the legs is formed in an articulated manner and interacts resiliently with the legs, so that a frame is formed is, and when the legs are compressed, an outwardly directed restoring force is generated, wherein the legs are designed in skeletal construction with multiple adjacent disk segments separated by slots, and arranged on the frame via webs.

Because of its elastic design it is possible to insert the disk-shaped augment in a cavity surrounded by the cortex at the bone head and sink it therein. In doing so, the outer surface of the disk-shaped augment abuts against the inside of the cortex, and the compression of the legs during insertion presses the advantageously porous structure on the outer sheath against the inside of the cortex. Owing to the preload force achieved in this way, an intimate connection is created between the outer sheath on the one hand and the cortex on the other.

Because of the skeletal construction, the disk-shaped augment is not designed as a unitary disk, but is divided into multiple adjacent disk segments. These disk segments typically have the same thickness. The disk segments are attached to the frame via webs, which is elastic due to the connecting piece interacting resiliently with the legs. This enables compression of the legs without causing tension between the disk segments. The skeletal construction enables particularly favorable compression and resiliency characteristics, which simplifies the insertion of the augment into a recess created on the bone head and ensures a more reliable fastening there. Surprisingly, if desired, there is also a sufficient sealing effect against bone cement, so that a continuous sealing plate is not necessary. Another advantage of skeletal construction is that augments of various size levels can easily be created by adding/enlarging or removing/reducing one or more disk segments, with typically a plurality, i.e. three or more disk segments, being provided. The skeletal construction with its numerous gaps, between the disk segments and towards the resilient frame, not only enables high compression, but also favors the ingrowth of bone material for long-term stable fastening.

The invention therefore combines two essential advantages. On the one hand, a favorable primary fixation is achieved and, on the other hand, the best conditions are created for the subsequent ingrowth of bone material and thus for a correspondingly solid, long-term stable fastening. In addition, if necessary, for explantation, the augment with its outer sheath can be separated from the surrounding bone structure by compression, which makes explantation easier.

Some terms used are explained below:

The compression of the legs is understood to mean a movement of the legs towards each other, narrowing a free space between them. This is also referred to below as deflecting. Correspondingly, a movement in the opposite direction is an expansion.

An anchoring keel of an endoprosthesis is understood to mean a structure that is provided for anchoring the endoprosthesis in a bone, in particular a medullary canal of the bone, and comprises a shaft and wing-like extensions arranged laterally thereon.

The first side of the augment is typically the one that is oriented inwards, i.e. towards the inside of the bone. The second side of the augment is typically the one that is oriented outwards in the opposite direction, i.e. towards the end of the bone.

The distance between the first and second sides of the disk segments determines the thickness of the augment. The frame may have a smaller thickness, but is preferably not thicker than the disk segments.

For contour-like objects, “undersized” is understood to mean that the outer contour of one object runs within the outer contour of the other object, preferably reduced in dimensions by a certain absolute or relative amount.

A ribbed structure is preferably understood to mean a coarsely ribbed structure, with a distance between individual profiles of the corrugation of at least one millimeter. It has been shown that such a coarse corrugation creates a reliable and long-term stable positive fastening to bone cement.

For the ribbed structure, sharp-edged is understood to mean that protruding areas of the corrugation are formed without rounded edges.

The porous structure preferably has a porosity in the range of 60% to 90% and/or an average pore size of 0.1 mm to 1.5 mm, in particular 0.4 mm to 1.0 mm. The thickness of the porous structure is preferably between 0.6 mm and 2.5 mm, the thickness being dimensioned such that at least one layer of pores located in the depth of the material (i.e. not lying on the surface or in section) is provided. As a result, undercut structures can be formed in the pores.

The first side of the disk-shaped augment is preferably designed as a porous structure. This creates favorable conditions for bone ingrowth and thus good fastening of the augment on the large contact surface with which the first side abuts flat against the bone. The edges are advantageously designed to be solid. Edges are understood to mean the transitions between side surfaces, in particular from the first side to lateral sides. This results in a reinforcement of the edges, which prevents the porous structure from breaking off.

The outer sheath is expediently designed as a porous structure that promotes bone ingrowth. This favors bone ingrowth. This means that the primary fixation caused by compression can be supported by long-term fixation through the ingrown bone material.

The porous structure is advantageously designed in such a way that it comprises connected pores in the depth of the material. The porosity therefore goes beyond superficial porosity and extends into the depths of the material. In this way, cavities are created that are connected to one another, which creates particularly favorable conditions for the ingrowth of bone material. The pores are advantageously dimensioned so that they have a width of about 0.4 mm to 1.0 mm. This results in particularly favorable ingrowth characteristic. Expediently, the porous structure comprises at least one and up to three layers of pores in the depth, which typically means a preferred thickness of the porous structure of about 0.6 mm to 2.5 mm. This results in undercut structures in the pores, which ensure a good holding effect in the event of ingrown bone material.

It is expediently provided that the porous structure has porous areas that are framed by a solid edge. This, on the one hand, achieves a clear limitation and, on the other hand, prevents undesirable breaking out of mechanically more sensitive porous material from the porous structure, especially in the edge area.

It is possible for the legs, in particular their disk segments, to be formed from a porous structure, with the second side expediently being provided with a cover plate. On the one hand, this results in a light-weight structure that, on the other hand, is almost completely open for the ingrowth of bone material. However, it is preferred that the legs have a solid (non-porous) core, on the outside of which pockets are formed in which the porous structure is arranged. This allows defined zones to be created where the porous structure is present and where the bone material is supposed to grow in. In addition, the legs or the augment become(s) mechanically more robust and resilient. Other advantages of pockets with limited depth in terms of porosity include easier production and easier cleaning.

The inner wall on the frame and/or on the disk segments is advantageously designed to be solid. This can prevent unwanted penetration of bone cement into the porous structure of the frame or of the disk segments. This also applies to lateral surfaces other than the outer sheath, e.g. this also applies to interior walls between which slots or gaps are formed.

Preferably, the slots are so narrow that they act as a gap seal for bone cement. Adequate sealing against bone cement is thus achieved in a simple manner with little effort. Preferably, the slots have a maximum width of 0.7 mm, more preferably a maximum width of 0.4 mm. Depending on the height of the disk segments and/or viscosity of the bone cement larger widths may suffice or smaller widths may be required to obtain an adequate sealing gap.

The lateral surfaces of the disk segments (usually the lateral outer sides or the side surfaces forming the slots) are advantageously designed to be solid, i.e. nonporous. This creates rigidity. Furthermore, in doing so, the risk of undesired penetration of bone cement into or through the slots is effectively minimized. With such a solid design of the lateral surfaces, the width of the slots is no longer relevant. The disk segments are preferably designed to be solid, if necessary, with pockets for porous structure on the outer sheath and/or on the first side.

Expediently, the frame is designed as a curved leaf spring. The tension resulting from the compression is distributed across the resilient frame, which leads to an even load and thus to a higher load capacity. It is particularly expedient if the frame is designed as an outer edge which encloses at least half of the entirety of the disk segments, and on the outside of which the outer sheath is arranged. The frame thus borders the entirety of the disk segments, which are located on the inside of the frame. Thus, the frame can simultaneously form the outer sheath with the porous structure.

The webs are advantageously designed to be flexible. This makes it possible for the disk segments to move relative to the frame. This opens up a lateral evasive movement of the respective disk segment, which can be particularly advantageous in the case of strong compression.

Preferably, at least one fastening hole for receiving a fastening screw is provided in the frame and/or the disk segments. In this way, an additional fastening of the augment to the bone can be achieved, which can make a particularly valuable contribution to primary fastening. Advantageously, the fastening hole has a solid perforated sheath. Such a solid perforated sheath provides rigidity in order to ensure the stability of the fastening hole even under the effect of the tightening force of the fastening screw. Furthermore, undesirable ingrowth of bone material can be prevented in this way.

For skeletal construction, it is expedient if the fastening hole is arranged in one of the disk segments. Preferably, this disk segment is not arranged on the resilient frame by means of a narrow web, but rather by means of a wider bridge, which, further preferably, is wider by at least half a web width (i.e. has a width of at least 1.5 times the width of a web). This achieves two advantages: on the one hand, an efficient fastening of the disk segment, which is additionally loaded by the fastening screw, is achieved. On the other hand, the wider bridge makes it possible to move the fastening hole closer to the edge and thus closer to the frame, which is a significant advantage, especially with small augment sizes, due to the improved use of space. A plurality of disk segments can also be provided with a fastening hole.

The through-opening is typically an elongated receiving space for an anchoring keel of the endoprosthesis. The through-opening can expediently be open towards one side. This means, it is located at the edge of the augment as an edge opening. This is particularly advantageous if the augment is designed as a half-sided augment. The term half-sided refers to the dimensions of the endoprosthesis, whose anchoring keel is guided through the through-opening of the augment. The endoprosthesis often only requires relining on one side, for example if a bone defect is limited to one side of the bone head and the augment is therefore only needed there. It should be understood that two half-sided augments can also be provided in order to provide relining on both sides.

The through-opening is typically rather small in relation to the dimensions of the augment; typically, it occupies less than half, often less than one third, of the volume of the augment. This has the advantage that still sufficient supporting material for the legs or the disk segments remains to ensure sufficient relining. The through-opening is usually dimensioned so that it can receive the anchoring keel or, if necessary, its wing-like extensions, with little clearance. The little clearance is expediently dimensioned to be large enough so that a desired compression, which leads to a reduction in the size of the through-opening due to the resilient deflection of the frame, is possible without hitting the anchoring keel. The invention can also include those augments that have a through opening, but not for an anchoring keel.

The outer sheath is advantageously not arranged at a 90° angle to the first side or to the second side. It has proven useful if the outer sheath is conically inclined, preferably tapering towards the first side at an angle of 5 to 10°. This achieves an adaptation to a tapering outer contour of the bone, and on the other hand, due to this conicity, the augment can be inserted or removed more easily by pressing than would be the case with a vertical outer sheath.

Outwardly directed spikes are expediently arranged on the outer sheath. When implanted, they press into the surrounding cortex of the bone head, in which the augment is received in a recess. This ensures a more reliable fastening right from the start, i.e. improved primary fixation. When the legs of the augment are pressed together (compression) for explantation, the spikes then automatically move back out of the cortex, thereby enabling explantation.

A cover plate can be provided on the second side, which has a ribbed structure as an interlocking for cement bonding. In this way, additional fastening can be achieved and a cutting layer is created, which, in the case of a planned explantation, can be cut through in a relatively simple manner using a saw, whereby the endoprosthesis is released from the augment and can therefore be easily removed without having to tear out an augment that may have grown heavily into the bone head. The ribbed structure is advantageously designed to be have grooves. Expediently, continuous grooves are provided, which are preferably oriented in a direction substantially transverse to the extent of the through-opening. The grooving achieves particularly effective interlocking with an applied cement bed. The grooves preferably have such spacing and height that is tailored to the flow and setting characteristic as well as the grain size of the cement bed. Groove spacings in the range of 2 to 10 mm and/or a groove depth of 0.5 to 2 mm have proven successful, with profiles delimiting the grooves being about 0.5 to 3 mm wide.

The ribbed structure can have sharp edges. However, other alternative profile shapes are also conceivable, alternatively triangular shapes or profile shapes with rounded corners.

Expediently, the ribbed structure is produced using a wire erosion process. In doing so almost any profile shape, including sharp-edged ones, can be formed efficiently.

It is particularly expedient if the augment is produced uniformly using an additive process, in particular 3D printing. In doing so even complex shapes in different sizes can be produced without or only with a minimum of complex post-processing. A combination with wire erosion is particularly expedient for creating defined, sharply delimited profiles, as mentioned above.

A cement-tight cover plate can be provided. Cement-tight is understood to mean that no bone cement passes through the cover plate. For this purpose, the cover plate can be designed with slot-like openings throughout. Bone cement applied to the top of the augment is separated from the inner and lower area of the augment so that no bone cement enters and interferes with ingrowth of bone material. The cover plate does not affect the resiliency characteristic of the frame including the compression and the relative mobility of the disk segments.

Advantageously, it is provided that the porous structure is provided with a coating that promotes bone ingrowth, in particular comprising calcium phosphate, and/or is provided with a biocidal coating, in particular comprising silver, on non-porous areas. This ensures that ingrowth of bone material is favored at desired places and that such ingrowth does not happen at other places where it is not desired, especially for the purpose of easier explantation.

Preferably, at least one flexible hinge is provided on the frame, which is designed to enable additional compression of the legs and to generate an outwardly directed restoring force. This creates—in addition to the leaf spring-like deflection of the frame as a whole—a localized flexible hinge, whereby a stronger compression is achieved, which is particularly effective even with lower force. In this way, a change in the spring stiffness of the elastic frame can be achieved. This is an advantage, especially for small augments, as they possibly would otherwise be too rigid due to their compactness. The term “localized” is understood here in the sense of spatially concentrated. Advantageously, a plurality of such localized flexible hinges can be provided in order to achieve a more even distribution of the additional compression and the application of force by the restoring force. A plurality of flexible hinges also favors largely true-to-form compression.

It is expedient that the at least one flexible hinge is formed by local material weakening, in particular in the form of a groove, slots and/or perforations. This means that flexible hinges can be created on the frame in a manner that is technically favorable. The design as a groove offers the advantage that the groove base maintains the separation of the interior and exterior space caused by the frame. Slots offer the advantage that a smaller bending rigidity can be set, providing protection against undesirable cement flow, if the slots are configured accordingly narrow (for preferred widths see above). The design as round perforations in particular offers the advantage that it can be produced efficiently and also has the advantage, if necessary, to enable subsequent adjustment by drilling additional holes. Round, especially circular perforations are therefore preferred. They also offer the advantage that they counteract load accumulation due to the lack of corners and therefore enable a favorable load distribution to the surrounding area of the flexible hinge. Several slots and in particular perforations can also be grouped together. In this way, even low restoring forces can be set, which is a considerable advantage, especially for small augments. —Adjustments can also be made through the choice of material, for example, a titanium alloy such as Ti6Al4V is more rigid than pure titanium (e.g. titanium Grade 2). Variations in width or depth of the groove or length and width of the remaining webs between the slots/perforations also enable adjustments in terms of the bending rigidity of the flexible hinges. Advantageously, if there is a plurality of flexible hinges, they can differ in terms of their material weakening, so that they have different restoring forces when compressed. This enables a finer setting of the bending rigidity, namely in terms of hardness and location.

The invention further includes an arrangement consisting of an endoprosthesis, in particular a knee joint endoprosthesis, and the disk-shaped augment. An anchoring keel of the endoprosthesis is received in the through-opening, particularly in the implanted state. The disk-shaped augment is expediently configured in its outer contour similar to the endoprosthesis, in particular a tibial plate. However, in comparison, said augment is undersized, i.e. it has somewhat smaller dimensions (particularly in the range of 2 to 6 mm along the outer sheath). As a result, it can support the endoprosthesis, especially the tibial plate, but nevertheless received in a recess bordered by the cortex at the bone head.

The disk-shaped augment is advantageously of half-sided design to support a left or right half of the endoprosthesis. As a result, effective support can be achieved by the augment if a bone defect is only present on one side of the bone head, thus avoiding the unnecessary removal of bone material on the other, healthy side.

The through-opening for receiving the anchoring keel and its wing-like extensions is expediently dimensioned with a defined free space of preferably 1 to 3 mm. This free space creates a clearance between the anchoring keel with its wing-like extensions and the legs of the augment, which enables tolerance compensation and the deflection of the frame through compression. In practice it is a considerable relief, as large compression and undesirable jamming of the augment and anchoring keel with its wing-like extensions can be avoided.

### BRIEF DESCRIPTION OF THE DRAWINGS

The invention is explained below with reference to the accompanying drawing using advantageous exemplary embodiments. In the drawing:

FIG. 1 shows a perspective view of a first exemplary embodiment for a disk-shaped augment;

FIG. 2A, B show a rear view and a medial sectional view of the augment according to FIG. 1;

FIG. 3A-F show different profile shapes for ribbing;

FIG. 4 shows a frontal view of the augment together with a tibial plate of a knee joint endoprosthesis;

FIGS. 5A, B show top views of a first side of the augment in two different sizes;

FIG. 6 shows an exploded view of the augment according to FIG. 1 with the knee joint endoprosthesis;

FIG. 7 shows a perspective view of the arrangement of the augment on the knee joint endoprosthesis;

FIG. 8 shows a top view of a first side of a second exemplary embodiment for a disk-shaped augment with flexible hinges; and

FIGS. 9A, B show detailed views of variants for setting a bending rigidity of the flexible hinges.

### DETAILED DESCRIPTION

The invention is explained below using an example of an augment for a knee joint endoprosthesis, specifically for an augment arranged on the upper (proximal) bone head of the tibia. The augment must be distinguished from the prosthesis, i.e. the augment is not an element of the actual prosthesis. The augment strengthens the bone and thus increases or improves its ability to accommodate the prosthesis. Augments according to the invention can of course also be provided at the other (distal) end or on other bones. Augment 1 preferably consists of a titanium alloy (e.g., Ti6Al4V) or pure titanium (e.g., titanium Grade 2), and can expediently be produced using an additive process (3D printing). However, it can also consist of other biocompatible material, for example metallic material such as, for example, cobalt-chromium-molybdenum (CoCrMo), stainless steel or plastic material, such as, for example, polyether ether ketone (PEEK).

Augment 1 of the exemplary embodiment explained here is provided to be arranged below tibial component 9 of a knee joint endoprosthesis, as shown in FIG. 4. The knee joint endoprosthesis comprises a femoral component (not shown) to be arranged on the femoral bone (not shown) and tibial component 9 to be arranged at the proximal end of a tibial bone 99. Said tibial component 9 comprises a tibial plate 91 which is arranged laterally extended on a resected bone head of the tibia. First (distal) side 92 of tibia plate 91 is designed to abut against the surface of the bone head of tibia 99. On the opposite second (proximal) side (top side) of tibia plate 91, a receptacle 90 is provided, in which a bearing piece (not shown) of the knee joint endoprosthesis is to be arranged. Depending on whether the tibial component is provided for cemented or cement-free implantation, tibia plate 91 is optionally provided on its first (distal) side 92 with a porous structure for the ingrowth of bone material in order to enable optimal fastening with cement-free implantation.

To attach tibia plate 91, an anchoring keel 8 is provided, which projects distally. It has a shaft piece 82 and a distally adjoining cone piece 81. Cone piece 81 is designed to, if necessary, to receive a plug-in shaft protruding into the medullary canal of tibia 9. Laterally, shaft piece 82 is adjoined by a wing-like extension 83 on both the left and right sides, at the free end of which a clamping sleeve 84 is arranged. The wing-like extensions 83 function as support arms for tibial plate 91. Tibia plate 91 can thus be connected to anchoring keel 8 at three points, that is to say centrally on shaft piece 82 and on each of the left and right sides with the clamping sleeves 84 arranged at the free ends of the wing-like extensions 83. For this purpose, two screw holes 94 are provided on the tibia plate 91 to the left and to the right, which are aligned with the respective clamping sleeve 84.

As FIG. 4 shows, the wing-like extensions 83 are not abutting flush against the bottom 92, but are held at a defined distance. For this purpose, a central support collar 93 is provided on bottom 92, against which shaft piece 82 abuts. Spacer inserts 96 with an external thread 97 are screwed into screw holes 94 (see FIG. 6), which create a defined distance from the clamping sleeves 84. In this way, together with the central support collar 93, a defined distance is set between bottom 92 of tibia plate 91 and wing-like extensions 83 of anchoring keel 8. This is highlighted there by the ellipse shown with a dotted line.

An exploded view is shown in FIG. 6. The main components that can be seen are augment 1 and tibial plate 91 with its anchoring keel 8. As already described above, installation or assembly involves attaching anchoring keel 8 with a shaft piece 82 to distal side 11 of tibial plate 91 using central screw 95. In addition, spacer inserts 96 made of titanium material with an external thread 97 are screwed into screw holes 94 and thus determine a defined distance between wing-like extensions 83 and distal side 92 of tibia plate 91. For additional fastening and support, fastening screws 98 are also provided, which are passed through the respective spacer insert 96 and which engage in an internal thread of respective clamping sleeve 84 and thus clamp it against the spacer insert 96. In this way, a defined distance is set between wing-like extensions 83 and distal side 92 of tibial plate 91.

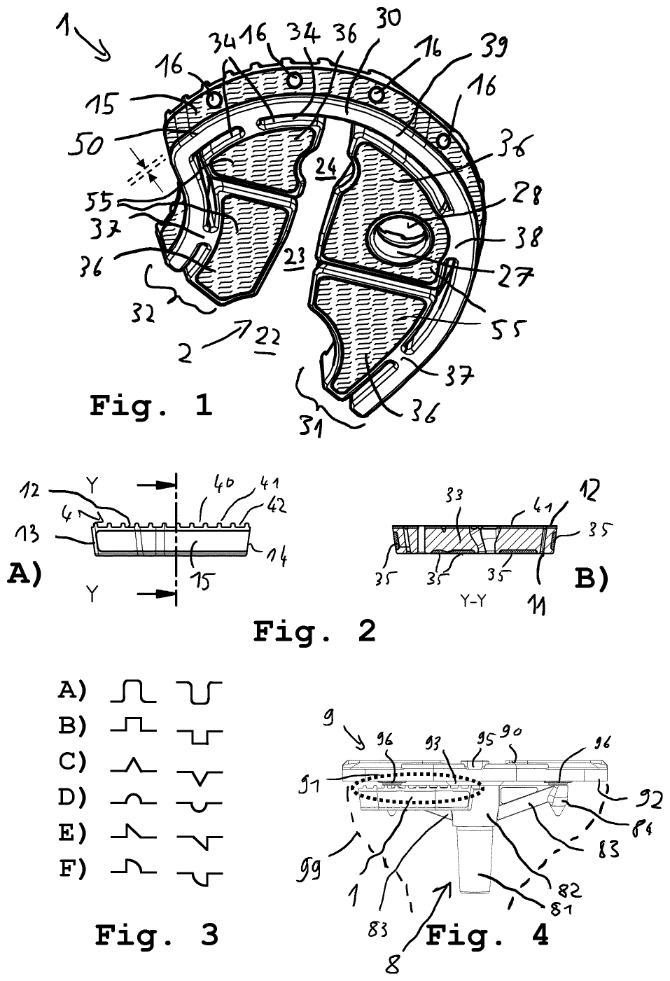

FIG. 1 shows a perspective view of first (distal) side 11 of a first exemplary embodiment of an augment 1 according to the present invention. Augment 1 is shaped like a thick disk and has two legs 31, 32 which are coupled resiliently in an articulated manner via a connecting piece 30 at the common end of legs 31, 32. An elastic frame 39 is thus formed, which typically encompasses about three-quarters of the circumference of the augment 1. Legs 31, 32 are divided into disk segments 36, with a through-opening 2 being arranged between legs 31, 32, which is designed to receive an anchoring keel 8 of tibial component 9. With frame 39 and disk segments 36, augment 1 is designed in a skeletal construction.

Disk segments 36 are each arranged on the inside of frame 39 via a web 37. Thus, narrow slots 34 are formed between disk segments 36 and between disk segments 36 and the inside of the wall of frame 39, which provide free space for a relative movement of disk segments 36 during compression/expansion. The width of slots 34 can be dimensioned such that a gap seal that is effective against the entry of bone cement is formed, taking into account the depth of the slots 34 predetermined by the thickness of disk segments 36 of augment 1. With these disk segments 36, a fine adjustment can be made to the space required by through-opening 2, which in turn is largely determined by the nature of anchoring keel 8 of the prosthesis to be implanted.

A rear view of augment 1 and a medial sectional view are shown in FIGS. 2A and B. The rear view refers to the orientation of augment 1 in the inserted state. Using anatomical terms, this is a posterior view. On top, i.e. in the exemplary embodiment on the second (proximal) side, a cement-tight cover plate 4 can be formed on augment 1. This does not interfere with the resiliency of frame 39 including the compression and the relative mobility of disk segments 36. The cover plate can be provided with a ribbed structure 40 on its top. The ribbed structure 40 can thus serve to interlock a cement bed (not shown), which fills the space between tibia plate 91 and thus provides additional fastening. Ribbing 40 can have grooves 42, the individual grooves being separated from one another by raised profiles. The grooves can be oriented in a direction from front to back, i.e. from anterior to posterior in relation to the implanted state. The depth of grooves 40 can, for example, be 1 mm, the width of the grooves about 3 to 4 mm and the width of raised profiles 41 about 1.5 mm. In the simplest case, profiles 41 can have a rectangular cross-sectional shape, as shown in FIG. 3A) or 3B), with rounded or sharp corners, each as a positive or negative profile. Alternatively, non-rectangular shapes for the profile are also possible, for example in the form of a symmetrical triangle or an asymmetrical, sawtooth-like triangle, as shown in FIG. 3C) or 3E). Furthermore, rounded profiles can also be provided, for example in the manner of a semicircle as shown in FIG. 3D) or asymmetrically in the manner of a quarter circle as shown in FIG. 3F). The person skilled in the art can make a selection depending on the requirements for the interlocking with the bone cement formed by profiles 41.

In FIGS. 2A, B it is apparent from the respective lateral sides of augment 1 that they taper conically towards the inside of the bone towards the first side (in the present case distally, in the figure downwards). In the exemplary embodiment shown, the angle on each side is about 7°. Other angles can also be provided. The conical configuration not only simplifies insertion during implantation, but above all it also simplifies removal of augment 1 from the bone during explantation. It can also be seen that frame 1 and disk segments 36 have about the same thickness.

Reference is now made to FIGS. 1 and 2B). As visualized by a dashed line in FIG. 1, first (distal) bottom 11 of augment 1 and an outer sheath 15 on lateral side 13 are each provided with a porous structure 5. In the embodiment shown, the porous structure is not continuous, but legs 31, 32 have a solid core 33. Pockets 35 are provided on the corresponding surfaces of legs 31, 32, in which porous structure 5 is arranged. Pockets 35 have a depth of about 0.8 to 2 mm. In FIG. 2B, such pockets are provided and shown on first (distal) side 11 and laterally on outer sheath 15. In order to prevent undesirable breakage of porous structure 5, particularly at the edges of augment 1, the edges between adjacent porous structures 5 at the transition from the distal side to the lateral side are provided with solid, i.e. non-porous, edges 50. Thus, reinforcement is achieved and porous structure 5 is prevented from breaking away. The width of these edges 50 is visualized by the two opposing arrows in FIG. 1 and is preferably about 1 mm to 2 mm.

Furthermore, spikes 16 pointing radially outwards can be arranged on outer sheath 15. After the implantation of augment 1, these spikes drill into the surrounding cortex of the tibial head and thus additionally secure augment 1 in its position.

Fastening hole 28 can be arranged in one of disk segments 36. It is dimensioned so that a cancellous bone screw 29 can be inserted through this opening and tightened as additional fastening. For additional stiffening, fastening hole 28 is provided with a solid perforated sheath 27 as an inner lining. Such a cancellous bone screw 29 for additional fastening is shown in FIG. 7.

In order to be able to bring fastening hole 28 closer to the edge of the disk segment, preferably close to surrounding frame 39, the web is expediently widened significantly at this point to form a bridge 38, which offers additional space so that fastening hole 28 can be arranged as close as possible to frame 39. This can be particularly advantageous for small sizes (but not limited to) in order to achieve the greatest possible distance from anchoring keel 8 and the bone cavity required to receive it for better support in the cancellous bone.

As can be clearly seen from FIG. 1, through-opening 2 is divided into multiple areas. The largest area at the edge of the through-opening is typically occupied by a receiving area 22 for shaft piece 82 of anchoring keel 8, with an elongated area 23 of the through-opening extending therefrom, which is typically designed to receive wing-like extension 83. At the far end thereof, there is typically a circular receiving area 24 which is provided for the clamping sleeves 84 at the free end of the respective wing-like extension 83. As can be seen in particular by a comparison with FIG. 7, through-opening 2 with its areas 22, 23 and 24 is matched to anchoring keel 8 in terms of dimensioning. As a result, anchoring keel 8 is received with clearance, which is visualized in FIG. 7 by the two opposing arrows. Its dimensions are so large that sufficient clearance to anchoring keel 8 with its extensions 83 remains and sufficient free space remains for compression of legs 31, 32 and frame 39 without disk segments 36 hitting anchoring keel 8. In the example shown in FIG. 7, it is about 1.5 to 2 mm.

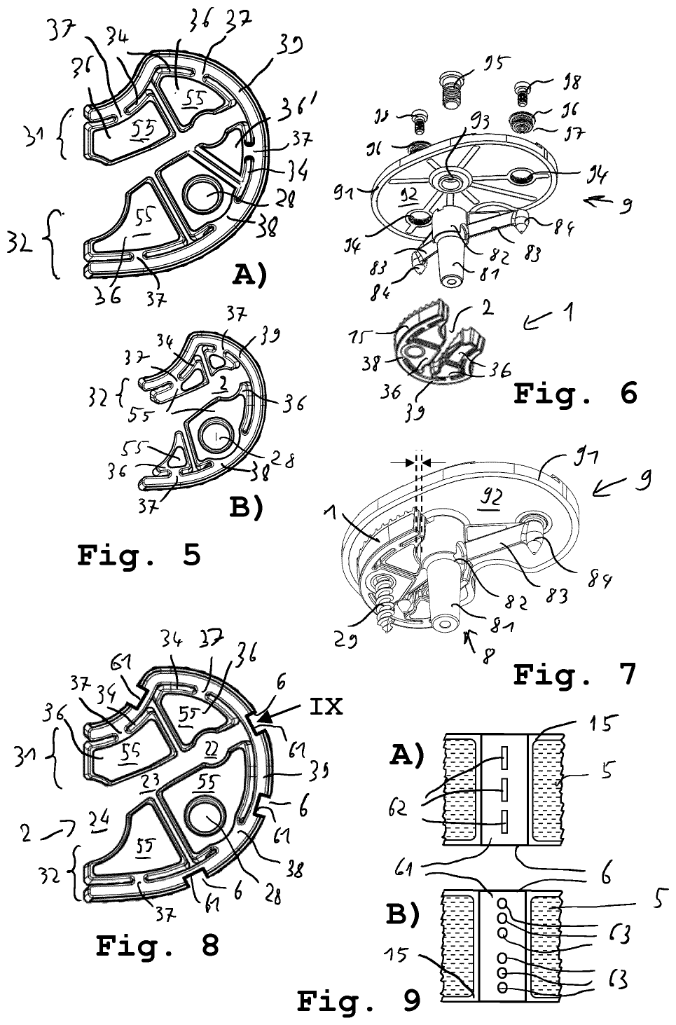

The skeletal construction with resilient frame 39 and disk segments 36 makes it possible to easily provide variants of the augment in larger or smaller sizes. The size and/or number of disk segments can be varied. For example, by omitting one of disk segments 36 or dimensioning disk segments 36 smaller, as shown in FIG. 5B), a smaller size of augment 1 can be formed. Or a larger size can be formed by adding a disk segment 36′, as shown in FIG. 5A), or by enlarging disk segments 36, as shown in FIG. 8. In this way, synergy effects arise when augments according to the invention are provided in an augment set with different sizes.

Porous structure 5 can be provided with a biocompatible coating 55, for example made of calcium phosphate, to further promote bone ingrowth. This applies to porous structure 5 of all embodiments.

Multiple flexible hinges 6 arranged on frame 39 are shown by way of example in FIG. 8. It should be understood that the flexible hinges can be arranged in other augments 1 shown. Flexible hinges 6 are formed on the outside of frame 39. They are each formed by a groove 61 running through outer sheath 15 from the first to the second side, so that in the corresponding area in relation to the material thickness only a relatively thin continuous strip remains. This results in a local material weakening. Flexible hinges 6 lead to a reduction in the bending rigidity of frame 39 overall. The circumference can be set by the type and number of flexible hinges 6.

A detailed view of one of flexible hinges 6 seen from the viewing direction, as shown by the arrow marked “IX”, is shown in FIGS. 9A, B). A piece of outer sheath 15 is shown in each case with a groove 61 in the center of the picture. Slots 62, as in FIG. 9A), or perforations 63, as in FIG. 9B), can optionally be provided. Slots 62 are preferably arranged in a line in order to achieve a defined bending line; however, this is not mandatory. The perforations are grouped like clusters in two groups 63′. Material areas remain in each case between slots 62 or perforations 63, the common width of which determining the bending rigidity of the respective flexible joint 6. The smaller the width, the less rigid is the flexible hinge 6 formed in this way. The round design of perforations 63 offers the advantage that it enables a particularly fine setting and furthermore achieves a favorable course of the mechanical load lines by avoiding sharp corners. In this way, a notch effect that is harmful to long-term stability can be reliably avoided.

The explantation of an endoprosthesis, a knee joint endoprosthesis, and disk-shaped augment 1 will be briefly explained below. Reference is made to FIG. 4, and to the saw slot shown there (see the area marked by the dashed ellipse), into which a saw can be inserted during an explantation. For explantation, fastening screws 98 are unscrewed in advance and spacer inserts 96 are also removed. The saw slot is now accessible from the side up to shaft piece 82 in the middle of tibia plate 91. The bottom of tibia plate 91 can thus be cut out using a saw (not shown). Finally, central screw 95 is removed and tibial plate 91 can now be removed. In this way, augment 1 with a cement layer possibly arranged thereon, is accessible, which cement layer can then also be removed if necessary. If desired, augment 1 can be explanted upwards by pressing legs 31, 32 together.

1. A disk-shaped augment for filling bone defects comprising:
a first side;
a second side;
an outer sheath on lateral sides, and
an inner wall for a through-opening running from the first side to the second side for an anchoring keel of an endoprosthesis arranged on the second side, wherein the augment is C-shaped with two legs flanking the through-opening, wherein
a connecting piece between the legs is formed in an articulated manner and interacts elastically with the legs so that a frame is formed, and when the legs are compressed an outwardly directed restoring force is generated, wherein the legs are designed in a skeletal construction with multiple adjacent disk segments separated by slots, and arranged on the frame via webs.

2. The disk-shaped augment according to claim 1, wherein the first side is designed as a porous structure, wherein edges are designed to be solid and/or the outer sheath is designed as a porous structure which promotes bony ingrowth.

3. The disk-shaped augment according to claim 2, wherein the porous structure comprises pores connected to one another in a depth of the material, wherein the pores comprise a width of 0.4 to 1.0 mm.

4. The disk-shaped augment according to claim 2, wherein the porous structure has porous areas that are framed by a solid edge.

5. The disk-shaped augment according to claim 2, wherein the legs have a solid core, on an outside of which pockets are formed, in which the porous structure is arranged.

6. The disk-shaped augment according to claim 1, wherein the legs are formed from a porous structure.

7. The disk-shaped augment according to claim 1, wherein the inner walls on the legs and the disk segments are solid.

8. The disk-shaped augment according to claim 1, wherein the slots are a narrow size that is configured to act as a gap seal for bone cement, having a maximum width of 0.7 mm.

9. The disk-shaped augment according to claim 1, wherein the frame is an outer edge which encloses at least half of the disk segments and on an outside of which the outer sheath is arranged.

10. The disk-shaped augment according to claim 1, wherein at least one fastening hole for receiving a fastening screw is provided in the frame and/or the disk segments, wherein the fastening hole has a solid perforated facia.

11. The disk-shaped augment according to claim 10, wherein the fastening hole is arranged in any one of the disk segments, wherein said disk segment is arranged on the frame by means of a bridge which is wider than the webs.

12. The disk-shaped augment according to claim 1, wherein the through-opening is an elongated receiving space for an anchoring keel and/or the through-opening is open on one side and/or the through-opening occupies less than half of a volume of the augment.

13. The disk-shaped augment according to claim 1, wherein an outer facia is conically inclined, tapering towards the first side at an angle of 5 to 10°.

14. The disk-shaped augment according to claim 1, wherein radially outwardly directed spikes are provided on an outer facia.

15. The disk-shaped augment according to claim 1, wherein a porous structure is provided with a bone ingrowth-promoting coating comprising calcium phosphate, and/or on non-porous areas with a biocidal coating.

16. The disk-shaped augment according to claim 1, wherein at least one flexible hinge is provided at the frame, which is configured to enable additional compression of the legs and to generate an outwardly directed restoring force.

17. The disk-shaped augment according to claim 16, wherein the at least one flexible hinge is formed by a local weakening of the material, in the form of a groove, a slot and/or perforations.

18. The disk-shaped augment according to claim 16, further comprising multiple flexible hinges, said flexible hinges differ in terms of their material weakening so that the multiple flexible hinges have restoring forces of varying levels upon compression.

19. An arrangement of a knee joint endoprosthesis and the disk-shaped augment according to claim 1, wherein an anchoring keel of the endoprosthesis is received in the through-opening.

20. The arrangement according to claim 19, wherein the disk-shaped augment is similar with respect to an outer contour and undersized in relation to the endoprosthesis.

21. The arrangement according to claim 19, wherein the disk-shaped augment is of half-sided design to support a left or right half of the endoprosthesis on one side.

22. The arrangement according to claim 19, wherein the through-opening is configured for receiving the anchoring keel and its wing-like extensions are dimensioned with a defined free space of 1 to 3 mm.
